(19) United States
(12) Patent Application Publication
(10) Pub. No.: US 2021/0032356 A1
(43) Pub. Date: Feb. 4, 2021

(54) METHOD FOR TREATING SEIZURES

(71) Applicant: Eastern Virginia Medical School, Norfolk, VA (US)

(72) Inventor: Alberto E. MUSTO, Norfolk, VA (US)

(21) Appl. No.: 16/945,709

(22) Filed: Jul. 31, 2020

Related U.S. Application Data
(60) Provisional application No. 62/882,152, filed on Aug. 2, 2019.

(51) Int. Cl. C07K 16/28; A61P 25/08

(52) U.S. Cl. CPC C07K 16/2875; A61P 25/08; A61K 2039/543; C07K 2317/40; C07K 2317/24

(57) ABSTRACT

Methods for treating seizures, more particularly treating seizures associated with epilepsy.

### CROSS-REFERENCE TO RELATED APPLICATIONS

This utility application claims priority to U.S. Provisional Application No. 62/882,152 filed Aug. 2, 2019, the contents of which are incorporated herein by reference in their entirety.

### FIELD OF THE INVENTION

This application relates to methods of treating seizures in a subject, more particularly to methods of preventing or treating seizures associated with temporal lobe epilepsy (TLE) by administering anti-CD40L antibodies to the subject.

### BACKGROUND

Temporal lobe epilepsy (TLE), or limbic epilepsy, is the most common form of focal epilepsy; there is currently no cure. The current medical treatments are not effective to control some limbic seizures. Patients with TLE have risk for early mortality and comorbidities such as cognitive dysfunctions, depression and anxiety disorders. Also, they have higher prevalence of systemic disorders that exacerbate adverse effects from anti-epileptic drugs. TLE is associated with social stigma; it also increases the costs of health care, often borne by the entire community.

TLE is a result of altered neuronal connectivity in the hippocampus. TLE is characterized by spontaneous recurrent complex partial seizures (limbic seizures) that arise in the hippocampus, spread within limbic circuitry and to other brain regions (Engel et al., 2012) as a product of underlying progressive complex biological events denominated limbic epileptogenesis (LE). LE is associated with brain injuries (Sloviter, 2008) that alter neuronal network connectivity which promotes hyper-excitable network and, limbic seizure susceptibility (Sutula et al., 1989; Musto et al., 2011). Other conditions such as traumatic brain injury, brain tumors, stroke, neuroinflammation, dementia, drug intoxication, chemical intoxications, neurodevelopmental disorders, and metabolic disorders of the nervous system, or a systemic disease such as diabetes, hypertension, chronic inflammatory disorders, and immunological disorders, can also increase the susceptibility for TLE, and for seizure and epilepsy in general.

There is a need for new and improved methods of preventing and/or treating seizures, in particular, seizures associated with temporal lobe epilepsy (TLE) or limbic epilepsy.

### BRIEF SUMMARY OF THE INVENTION

In accordance with one aspect, the invention provides a method for treating seizures in a subject in need thereof, including administering to the subject a therapeutically effective amount of an agent that inhibits the CD40-CD40L interaction and pathway in the brain.

In accordance with certain embodiments, the subject may have increased susceptibility for epilepsy.

In accordance with certain embodiments, the increased susceptibility for epilepsy may be due to a condition selected from the group consisting of traumatic brain injury, brain tumors, stroke, neuroinflammation, dementia, drug intoxication, chemical intoxications, neurodevelopmental disorders, and metabolic disorders of the nervous system. In accordance with certain other embodiments, the increased susceptibility for epilepsy may be due to a systemic disease selected from the group consisting of diabetes, hypertension, chronic inflammatory disorders, and immunological disorders.

In accordance with certain embodiments, the subject may manifest epilepsy. In accordance with certain other embodiments, the subject may not manifest epilepsy. In accordance with certain embodiments, the epilepsy is Temporal Lobe Epilepsy (TLE). In accordance with certain other embodiments, the epilepsy is status epilepticus.

In accordance with certain embodiments, the subject may be a mammal. In accordance with certain embodiments, the subject may be a human.

In accordance with certain embodiments, the agent that inhibits the CD40-CD40L interaction and pathway in the brain may be an antagonist of CD40 or CD40L. In accordance with certain embodiments, the antagonist may be a CD40L antagonist. In accordance with certain embodiments, the CD40L antagonist may be an anti-CD40L antibody.

In accordance with certain embodiments, the anti-CD40L antibody includes a fully humanized anti-CD40L antibody. In accordance with certain embodiments, the anti-CD40L antibody includes a fragment of a fully functional anti-CD40L antibody. In accordance with certain embodiments, the anti-CD40L antibody may be PEGylated.

In accordance with certain embodiments, the anti-CD40L antibody may be administered in a pharmaceutically acceptable composition. In accordance with certain embodiments, the anti-CD40L antibody may be administered by transdermal, transmucosal, intravenous, intramuscular, subcutaneous, intrathecal, intracerebral, intraarterial, intracisternal, endovenous, intraocular, oral and intradermal routes.

In accordance with certain embodiments, the anti-CD40L antibody may be administered by the transmucosal route. In accordance with certain embodiments, the anti-CD40L antibody may be administered intranasally. In accordance with certain embodiments, the anti-CD40L antibody is administered as nasal drops or a nasal spray.

In accordance with certain embodiments, seizures are prevented from occurring after the subject is treated. In accordance with certain other embodiments, the severity of seizures is decreased after the subject is treated. In accordance with certain embodiments, the frequency of seizures is decreased after the subject is treated.

### BRIEF DESCRIPTION OF THE DRAWINGS

FIG. 1A: Cresyl violet staining of the hippocampal region from a Temporal Lobe Epilepsy (TLE) patient, showing the dentate gyrus (DG) region.

FIG. 1a: Magnification of the box in FIG. 1A.

FIG. 1B: Cresyl violet staining of the hippocampal region from a Temporal Lobe Epilepsy (TLE) patient, showing the CA3 region.

FIG. 1b: Magnification of the box in FIG. 1B.

FIG. 1C: Cresyl violet staining of the hippocampal region from a Temporal Lobe Epilepsy (TLE) patient, showing the CA3 region.

FIG. 1c: Magnification of the box in FIG. 1C.

FIG. 1D: A representation of CD40 immunoreactivity in a temporal lobe specimen from a TLE patient.

FIG. 1E: A semi-quantification scale to describe CD40 IR.

FIG. 1F: CD40 IR in the hippocampal tissue of four different TLE patients.

FIG. 1G: CD40 immunoreactivity in sections from the CA1-subicular region of different TLE patients. The white arrowheads in G point to CD40 IR signals in cell bodies of likely neurons.

FIG. 1H: CD40 immunoreactivity in sections from the CA1 region of different TLE patients. The black arrowheads in H point to CD40 IR signals in cell bodies of astrocytes and neurons.

FIG. 2A: Cross sections of non-epileptic human brain, adapted from the The Human Protein Atlas (https://www.proteinatlas.org).

FIG. 2A-1: cross section of a healthy human hippocampus.

FIG. 2A-2: Astrocytes in a healthy human brain, stained with an antibody for Glial fibrillary acidic protein (GFAP). The arrow in FIG. 2A2 points to the cell body of an astrocyte.

FIG. 2A-3: CD40 staining in neurons from a patient with glioblastoma multiforme (GBM). The arrow points to the cell body of a neuron.

FIG. 2B-a: Representative sections of hippocampus from a TLE patient with CD40 immunoreactivity (arrows) in astrocytes.

FIG. 2B-b: Representative sections of hippocampus from a TLE patient with CD40 immunoreactivity (arrows) in astrocytes.

FIG. 2B-c: Representative sections of hippocampus from a TLE patient with CD40 immunoreactivity (arrows) in microglia.

FIG. 2B-d: Representative sections of hippocampus from a TLE patient with CD40 immunoreactivity (arrows) in oligodendrites.

FIG. 2B-e: Representative sections of hippocampus from a TLE patient with CD40 immunoreactivity (arrows) in neurons.

FIG. 2B-f: Representative sections of hippocampus from a TLE patient with CD40 immunoreactivity (arrows) in neurons.

FIG. 3A-a: Cresyl violet staining of a coronal section of the dorsal hippocampus of a wild type mouse with no seizure history.

FIG. 3A-b: CD40 IR in CD40KO mice.

FIG. 3A-c: CD40 IR in WT mice.

FIG. 3B-a: an overview of CD40 IR in the CA1 region of a naïve (no seizure experience) adult mouse

FIG. 3B-b: CD40 IR in the pyramidal layer of CAL Arrow points to cells demonstrating CD40 immunoreactivity.

FIG. 3B-c: CD40 IR in the Motor Cortex. Arrow points to cells demonstrating CD40 immunoreactivity.

FIG. 3B-d: CD40 IR in the Somatosensory Cortex. Arrow points to cells demonstrating CD40 immunoreactivity.

FIG. 3B-e: CD40 IR in the Lacunosum Moleculare Layer, magnification from the lower left square in 3B-a. Arrows point to cells demonstrating CD40 immunoreactivity.

FIG. 3B-f: CD40 IR in the Stratum Radiatum, magnification from the upper right square in 3B-a. Arrows point to cells demonstrating CD40 immunoreactivity.

FIG. 4: Expression of CD40 in cortex and hippocampus in WT mice.

FIG. 5A: Expression of CD40 in pre-synaptic and post-synaptic terminals in mouse hippocampus.

FIG. 5B: CD40 expression increases in mouse CA3 and Cortex after seizure. The arrows point to exemplary neurons shown to express CD40.

FIG. 5C: Semi-quantitative analyses of CD40 expression in different mouse hippocampal regions after seizure.

FIG. 5D: CD40L expression increase in the mouse hippocampus after seizure.

FIG. 5E: CD40L expression increase in the mouse cortex after seizure.

FIG. 6A: CD40 and CD40L expression increases in mice after status epilepticus.

FIG. 6B: Change of PP38/P38 in mice after status epilepticus.

FIG. 7A-a: A photograph of a C57BL/6 mouse showing a silicon probe.

FIG. 7A-b: An example of a silicon probe design showing the assembled 16 microelectrodes and their local field potentials.

FIG. 7A-c: An example of a Neuronexus smart box.

FIG. 7B-a: Monitoring overall oscillatory activity using power spectral analysis.

FIG. 7B-b: Monitoring overall oscillatory activity using power spectral analysis.

FIG. 8A: Recording LFP in mice during Status Epilepticus.

FIG. 8B: An example of a simultaneous recording of behavior and LFPs.

FIG. 8C: Musto's score used to assess the clinical condition of post seizure mice.

FIG. 8D: Spontaneous seizures and hyper-activity after status epileptics (SE). CD40KO mice post SE, WT mice post SE, and WT naïve (without prior seizure) mice were observed.

FIG. 9A: CD40KO mice presented reduced seizure severity after PTZ administration compared to WT mice.

FIG. 9B: CD40KO mice exhibited increased latency after PTZ administration compared to WT mice.

FIG. 9C: CD40KO mice exhibited lower seizure activity after PTZ administration.

FIG. 9D: CD40KO mice exhibited lower mortality after PTZ administration.

FIG. 10: CD40 deficiency in mice limits severity of seizures.

FIG. 11: Neuron loss in mice after status epilepticus.

FIG. 12A: Expression of CD40 after seizure in both WT and CD40KO (negative control) mice.

FIG. 12B: In the CA3 region of wild type mice, CD40 is upregulated to a greater degree after tonic-clonic generalized seizures than after partial seizures. The arrows in the figure for generalized seizure point to areas of especially high IR.

FIG. 12C: The distribution of CD40 IR in the mouse hippocampus.

FIG. 13A: The experimental design to test the effect of intranasal administration of anti-mouse CD40L on seizure susceptibility.

FIG. 13B: The administration of anti-CD40L to mice reduces Racine's score after 3rd dose of PTZ (p=0.02).

FIG. 13C: The administration of anti-CD40L to mice reduces the percentage of animals that reach Racine's score stage 4 after the 3rd dose of PTZ. (p=0.03).

FIG. 14A: The experimental design to test the effect of intranasal administration of anti-mouse CD40L on severe seizure.

FIG. 14B: The administration of anti-CD40L in mice reduces the percentage of animals that reach Racine's score stage 5 (p=0.03).

FIG. 14C: The administration of anti-CD40L in mice increases (p=0.01) latency for stage 3 seizure. (p=0.01).

### DETAILED DESCRIPTION OF THE INVENTION

The terms “treatment,” “treating,” “treat,” “therapy,” “therapeutic,” and the like are used herein to refer generally to obtaining a desired pharmacological and/or physiological effect, in humans and/or animals. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, and includes: (a) preventing the disease or symptom from occurring in a subject which may be predisposed to the disease or symptom, may or may not be diagnosed as having it; (b) inhibiting the disease symptom, i.e., arresting its development; or (c) relieving the disease symptom, i.e., causing regression of the disease or symptom.

The term “pharmaceutically acceptable carrier,” as used herein, refers to any and all solvents, dispersion media, coatings, antibacterial and antifungal agent, isotonic and absorption delaying agents for pharmaceutical active substances as are well known in the art. The term “pharmaceutical” or “agent”, as used herein, includes biological pharmaceuticals such as proteins, peptides, and oligonucleotides. Except insofar as any conventional media or agent is incompatible with the agent, its use in the therapeutic pharmaceutical compositions is contemplated. Supplementary compounds or biological pharmaceuticals can also be incorporated into the pharmaceutical compositions.

As used herein, the term “excipient” refers to the additives used to convert a synthetic agent into a form suitable for its intended purpose. For pharmaceutical compositions of the present invention suitable for administration to a human, the term “excipient” includes those excipients described in the HANDBOOK OF PHARMACEUTICAL EXCIPIENTS, American Pharmaceutical Association, 2nd Ed. (1994), which is herein incorporated in its entirety. The term “excipients” is meant to include fillers, binders, disintegrating agents, lubricants, solvents, suspending agents, dyes, extenders, surfactants, auxiliaries and the like. Liquid excipients can be selected from various oils, including those of petroleum, animal, vegetable or synthetic origin, such as, peanut oil, soybean oil, mineral oil, sesame oil, hydrogenated vegetable oil, cottonseed oil, groundnut oils, corn oil, germ oil, olive oil, or castor oil, and so forth.

Suitable excipients also include, but are not limited to, fillers such as saccharides, lactose, fructose, sucrose, inositol, mannitol or sorbitol, xylitol, trehalose, cellulose preparations and/or calcium phosphates, tricalcium phosphate or calcium hydrogen phosphate, as well as starch paste, using modified starch, maize starch, wheat starch, rice starch, potato starch, gelatin, tragacanth, ethoxylated isostearyl alcohols, polyoxyethylene sorbitol and sorbitan esters, microcrystalline cellulose, hydroxypropyl cellulose, methyl cellulose, hydroxypropyl methyl cellulose, aluminum metahydroxide, bentonite, sodium carboxymethylcellulose, croscarmellose sodium, crospovidone and sodium starch glycolate, and/or polyvinyl pyrrolidine and mixtures thereof. If desired, disintegrating agents can be added, such as, the above-mentioned starches and also carboxymethyl-starch, cross-linked polyvinyl pyrrolidone, agar, or alginic acid or a salt thereof, such as, sodium alginate. Auxiliaries include, silica, stearic acid or salts thereof, such as, magnesium stearate, sodium stearyl fumarate, or calcium stearate.

The expression “therapeutically effective amount” refers to an amount of an agent disclosed herein, that is effective for preventing, ameliorating, treating or delaying the onset of a disease or condition, in humans and/or animals.

The pharmaceutical compositions of the inventions can be administered to any animal that can experience the beneficial effects of the agents of the invention. Such animals include humans and non-humans such as primates, pets and farm animals.

### Pharmaceutical Compositions Comprising Agents of the Invention

The present invention also comprises pharmaceutical compositions comprising the agents disclosed herein. Routes of administration and dosages of effective amounts of the pharmaceutical compositions comprising the agents are also disclosed. The peptides of the present invention can be administered in combination with other pharmaceutical agents in a variety of protocols for effective treatment of disease, in humans and/or animals.

The pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired.

In addition to the agents disclosed herein, the pharmaceutical compositions of the present invention may further comprise at least one of any suitable auxiliaries including, but not limited to, diluents, binders, stabilizers, buffers, salts, lipophilic solvents, preservatives, adjuvants or the like. Pharmaceutically acceptable auxiliaries are preferred. Examples and methods of preparing such sterile solutions are well known in the art and can be found in well-known texts such as, but not limited to, REMINGTON'S PHARMACEUTICAL SCIENCES (Gennaro, Ed., 18th Edition, Mack Publishing Co. (1990)). Pharmaceutically acceptable carriers can be routinely selected that are suitable for the mode of administration, solubility and/or stability of the agent.

Pharmaceutical excipients and additives useful in the present invention can also include, but are not limited to, proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination in ranges of 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

Carbohydrate excipients suitable for use in the present invention include monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), myoinositol and the like.

Pharmaceutical compositions suitable for parenteral administration include aqueous and non-aqueous sterile injection solutions which may contain anti-oxidants, buffers, bacteriostats and solutes that render the formulation isotonic with the blood of the intended recipient; and aqueous and non-aqueous sterile suspensions which may include suspending agents and thickening agents. The pharmaceutical compositions may be presented in unit-dose or multi-dose containers, sealed ampules and vials, and may be stored in a freeze-dried (lyophilized) condition requiring only the addition of the sterile liquid carrier, water for injections, immediately prior to use. Extemporaneous injection solutions and suspensions may be prepared from sterile powders, granules and tablets of the kind previously described.

For parenteral administration, sterile suspensions and solutions are desired. Isotonic preparations which generally contain suitable preservatives are employed when intravenous administration is desired. The pharmaceutical compositions may be administered parenterally via injection of a pharmaceutical composition comprising an agent dissolved in an inert liquid carrier. The term “parenteral,” as used herein, includes, but is not limited to, subcutaneous injections, intravenous, intramuscular, intraperitoneal injections, or infusion techniques. Acceptable liquid carriers include, vegetable oils such as peanut oil, cotton seed oil, sesame oil and the like, as well as organic solvents such as solketal, glycerol formal and the like. The pharmaceutical compositions may be prepared by dissolving or suspending the agent in the liquid carrier such that the final formulation contains from about 0.005% to 30% by weight of an agent.

The composition of the invention can also include additional therapeutic agents such as, but not limited to hydrophilic drugs, hydrophobic drugs, hydrophilic macromolecules, cytokines, peptidomimetics, peptides, proteins, toxoids, sera, antibodies, vaccines, nucleosides, nucleotides, nucleoside analogs, genetic materials and/or combinations thereof.

In addition to agents and pharmaceutical compositions of the invention, and additional pharmaceutically active agents, the pharmaceutical formulation can also contain suitable pharmaceutically acceptable carriers comprising excipients and auxiliaries that facilitate processing of the active agents into preparations that can be administered to animals, as described herein.

Pharmaceutical formulations useful in the present invention can contain a quantity of as agent(s) according to this invention in an amount effective to treat the condition, disorder or disease of the subject being treated.

The invention is also directed to a kit form useful for administration to patients in need thereof. The kit may have a carrier means being compartmentalized in close confinement to receive two or more container means therein, having a first container means containing a therapeutically effective amount of a pharmaceutical composition of the invention and a carrier, excipient or diluent. Optionally, the kit can have additional container mean(s) comprising a therapeutically effective amount of additional agents.

The kit comprises a container for the separate pharmaceutical compositions such as a divided bottle or a divided foil packet, however, the separate pharmaceutical compositions can also be contained within a single, undivided container. Typically, the kit contains directions for administration of the separate components. The kit form is particularly advantageous when the separate components are preferably administered at different dosage intervals, or when titration of the individual components of the combination is desired by the prescribing physician. The kits of the invention include testing and screening kits and methods, to enable practitioners to measure levels of the active ingredients in bodily fluids. The kits of the invention also include research-grade reagents and kits available for use and purchase by research entities.

### Routes of Administration of Pharmaceutical Compositions Comprising the Agents of the Invention

The invention further relates to the administration of at least one agent disclosed herein by the following routes, including, but not limited to oral, parenteral, subcutaneous, intramuscular, intravenous, intrarticular, intrabronchial, intraabdominal, intracapsular, intracartilaginous, intracavitary, intracelial, intracelebellar, intrathecal, intracerebral, intraarterial, intracisternal, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarenal, intraretinal, intraspinal, intrasynovial, intrathoracic, intrauterine, endovenous, intradermal, intraocular, intravesical, bolus, vaginal, rectal, buccal, sublingual, intranasal, iontophoretic means, or transdermal means.

The administration in this invention can be through the airway. Administration per airway includes, for example, nasopharyngeal, oropharyngeal, and sometimes endotracheal.

The administration in this invention can be transmucosal administration. Transmucosal administration includes, for example, at least intranasal, sublabial, sub- or supralingual or buccal administration. Other methods of transmucosal administration may also be used.

### Temporal Lobe Epilepsy

Temporal lobe epilepsy (TLE), or limbic epilepsy, is the most common form of focal epilepsy. It is a result of altered neuronal connectivity in the hippocampus. TLE is characterized by spontaneous recurrent complex partial seizures (limbic seizures) that arise in the hippocampus, spread within limbic circuitry and to other brain regions (Engel et al., 2012) as a product of underlying progressive complex biological events denominated limbic epileptogenesis (LE). LE is associated with brain injuries (Sloviter, 2008) that alter neuronal network connectivity which promotes hyper-excitable network and, limbic seizure susceptibility (Sutula et al., 1989; Musto et al., 2011). Other conditions such as traumatic brain injury, brain tumors, stroke, neuroinflammation, dementia, drug intoxication, chemical intoxications, neurodevelopmental disorders, and metabolic disorders of the nervous system, or a systemic disease such as diabetes, hypertension, chronic inflammatory disorders, and immunological disorders, can also increase the susceptibility for TLE, and for seizure and epilepsy in general.

It is important to provide treatment for epilepsy and seizures, especially when the susceptibility for seizures and epilepsy is increased. The terms “treatment” and “treating” as used herein to refer generally to obtaining a desired pharmacological and/or physiological effect, and includes: (a) preventing the disease or symptom from occurring in a subject which may be predisposed to the disease or symptom, and who may or may not be diagnosed as having it; (b) inhibiting the disease symptom, i.e., arresting its development; and/or (c) relieving the disease symptom, i.e., causing regression of the disease or symptom. After treatment for epilepsy or seizures, seizures may be entirely prevented and/or the severity or frequency of the seizures may decrease. In this invention, “treatment” or “treating” epilepsy encompasses both in humans and in animals.

CD40 is expressed at a very low level in normal human hippocampus. It is therefore unexpected and surprising that genetic deficiency in CD40 or intranasal administration of anti-CD40L antibody limited seizure susceptibility, and reduced the frequency and severity of acute seizures induced by PTZ.

### Methods of Preparation of Pharmaceutical Compositions of the Present Invention

Methods of preparing various pharmaceutical compositions with a certain amount of active ingredients are known, or will be apparent in light of this disclosure, to those skilled in the art. Methods of preparing said pharmaceutical compositions can incorporate other suitable pharmaceutical excipients and their formulations as described in Remington's Pharmaceutical Sciences, Martin, E. W., ed., Mack Publishing Company, 19th ed. (1995).

One of ordinary skill in the art will appreciate that a method of administering pharmaceutically effective amounts of the pharmaceutical compositions of the invention to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Dosages can also be administered in a patient-specific manner to provide a predetermined concentration of the agents in the blood, as determined by techniques accepted and routine in the art. Dosage regimens are adjusted to provide the desired response, e.g., a therapeutic response or a combinatorial therapeutic effect. Generally, doses of the anti-CD40L antibody can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In some embodiments, a subject in need of treatment with an anti-CD40L antibody is administered the antibody at a dose 2 mg/kg, 3 mg/kg, 4 mg kg, 5 mg/kg, 10 mg/kg, 15 mg/kg, 20 mg/kg, 30 mg/kg, 35 mg kg, or 40 mg/kg.

A composition may comprise about 1 mg/mL to 100 mg/ml or about 10 mg/mL to 100 mg/mL or about 50 to 250 mg/mL or about 100 to 150 mg/mL or about 100 to 250 mg/mL of anti-CD40 antibody or antigen-binding fragment thereof.

### CD40 and CD40L

CD40 is a Type 1 transmembrane receptor expressed by B cells, macrophages, dendritic cells, and other cell types, including platelets, epithelial, endothelial, and stromal cells. The engagement of CD40 by its ligand, CD40 ligand (CD40L also known as CD 154), constitutes a key axis for the activation of innate and adaptive immune functions. CD40 and CD40L are described in various publications, such as WO 2016/028810 and U.S. Pat. No. 7,510,711, the contents of which are hereby incorporated by reference. In accordance with one aspect, the present application is directed to methods for blocking CD40-CD40L interaction and potential downstream pathway using an antibody administered (e.g., systemically or intraperitoneally) to prevent the onset of temporal lobe epilepsy. Blocking CD40L-CD40 interaction and potential downstream pathway may also be effective to treat neurological disorders with disruptive neuronal networks e.g. Alzheimer's disease and post-traumatic stress disorder.

CD40 is commonly expressed on immune cells such as monocytes and dendritic cells, but it is also expressed on non-immunological cells such as neurons, microglia, and endothelium. Additionally, CD40 contributes to the post-injury inflammatory environment. Microglial activation by Lipopolysaccharide (LPS) has been shown to increase expression of CD40 and CD40L secretion, and this upregulation increases the secretion of the pro-inflammatory cytokines TNF-α, IL-1β and IL-6.

In some cases, CD40 mimics and/or act synergistically with the function of the pro-inflammatory cytokine Tumor Necrosis Factor alpha (TNF-α) which plays a role in acute seizures. TNF-α, released from physiologically activated microglia and astrocytes, contributes to the homeostatic level of glutamate via TNF receptor 1 (TNFR1) and regulates formation and organization of excitatory and inhibitory synapses. Following an injury, TNF-α up-regulates AMPA receptors, augmenting glutamatergic transmission causing neurotoxicity and hyper-excitability exacerbated by induction of GABA receptor endocytosis, which reduces the inhibitory drive.

CD40 is expressed at a very low level in normal human hippocampus. It is therefore unexpected and surprising that genetic deficiency in CD40 or intranasal administration of anti-CD40L antibody limited seizure susceptibility, and reduce the frequency and severity of acute seizures induced by PTZ.

### Anti-CD40L Antibody

The term “antibody” is used herein in the broadest sense and covers fully assembled antibodies, antibody fragments which retain the ability to specifically bind to the CD40 antigen (e.g., Fab, Fv, and other fragments), single chain antibodies (scFv), diabodies, bispecific antibodies, chimeric antibodies, humanized antibodies, fully human antibodies, and the like, and recombinant peptides comprising the foregoing. The term “antibody” covers both polyclonal and monoclonal antibodies.

As used herein “anti-CD40L antibody” encompasses any antibody that specifically recognizes the CD40 ligand (CD40L) antigen. In some embodiments, anti-CD40L antibodies for use in the methods of the present invention, including monoclonal anti-CD40L antibodies, exhibit a strong single-site binding affinity for the CD40L antigen. Such monoclonal antibodies exhibit an affinity for CD40L (KD) of at least 10⁻⁵ M, preferably at least 10⁻⁶ M, at least 10⁻⁷ M, at least 10⁻⁸ M, at least 10⁻⁹ M, at least 10⁻¹⁰ M, at least 10⁻¹¹ M or at least 10⁻¹² M, when measured using a standard assay such as Biacore™. Biacore analysis is known in the art and details are provided in the “BIAapplications handbook”. The anti-CD40L antibodies for use in the methods of the present invention can be produced using any suitable antibody production method known to those of skill in the art.

The anti-CD40L antibody used in the methods of the present invention may be a monoclonal antibody. The term “monoclonal antibody” (and “mAb”) as used herein refers to an antibody obtained from a substantially homogeneous population of antibodies, i.e., the individual antibodies comprising the population are identical except for possible naturally occurring mutations that may be present in minor amounts. The term is not limited regarding the species of the antibody and does not require production of the antibody by any particular method. In contrast to polyclonal antibody preparations, which typically include different antibodies directed against different antigenic determinants (epitopes), each monoclonal antibody is directed against a single determinant (epitope) on the antigen.

The term “monoclonal” as originally used in relation to antibodies referred to antibodies produced by a single clonal line of immune cells, as opposed to “polyclonal” antibodies that, while all recognizing the same target protein, were produced by different B cells and would be directed to different epitopes on that protein. As used herein, the word “monoclonal” does not imply any particular cellular origin, but refers to any population of antibodies that all have the same amino acid sequence and recognize the same epitope in the same target protein. Thus, a monoclonal antibody may be produced using any suitable protein synthesis system, including immune cells, non-immune cells, acellular systems, etc.

In some embodiments, fully human antibodies to CD40L, for example, are obtained by immunizing transgenic mice. One such mouse is obtained using XenoMouse® technology (Abgenix; Fremont, Calif.), and is disclosed in U.S. Pat. Nos. 6,075,181, 6,091,001, and 6,114,598. For example, to produce the HCD122 antibody (commercially available as Lucatumumab), mice transgenic for the human IgG1 heavy chain locus and the human κ light chain locus were immunized with Sf9 cells expressing human CD40. Mice can also be transgenic for other isotypes.

Anti-CD40L antibody can be administered as a therapeutic agent for prevention, prophylaxis, or other therapy of epilepsy, such as TLE. The anti-CD40L antibody protein can be administered per intracerebral, intraventricular or intracisternal routes to avoid systemic delivery methods that require higher dosages. The pharmaceutical compositions disclosed herein may be administered in the form of, for example, pharmaceutically acceptable salts, or in the form of PEGylated protein compositions. PEGylation of protein therapeutics is well known in the art. PEGylation helps increase the circulation half-life of proteins by reducing their renal clearance rates. Other potential positive effects include enhanced solubility, improved stability, sustained absorption, and reduced immunogenicity, antigenicity and proteolysis. PEGylation may be accomplished by the random conjugation of linear polyethylene glycol chains onto the functional groups along the protein backbone or by the use of branched PEG (polyethylene glycol), or site-specific and controlled PEGylation. PEGylation techniques and the administration of PEGylated protein pharmaceutical compositions are within the level of skill in the art.

Moreover, the protein can be adapted for administration by any appropriate route, for example by the oral, nasal, topical (including buccal, sublingual, or transdermal), inhalational or parenteral (including subcutaneous, intracutaneous, intramuscular, intraarticular, intraperitoneal, intrasynovial, intrasternal, intrathecal, intralesional, intravenous, or intradermal injections or infusions) route. For human administration, the formulations preferably meet sterility, pyrogenicity, general safety, and purity as required by FDA Office and Biologics standards.

Dosage amounts of and modifications to the anti-CD40L antibody protein may be tissue, organ, and/or patient specific. For example, the exact dosage amount of and/or modification to the anti-CD40L antibody can be guided by expression patterns of the protein in a given tissue to avoid side effects or to enhance therapeutic effect. The selection of a dosage amount or modification of the protein may be dependent upon the specific type of disease sought to be treated, or the stage of the disease. The selected dosage amount is a therapeutically effective amount, or an amount sufficient to retard or arrest further seizures. The effect of a certain amount of the pharmaceutical composition can be monitored by observing the changes in the frequency and severity of seizures. Determining a therapeutically effective amount is well within the skill of a practicing physician. Accordingly, it may be necessary for the therapist to titer the dosage and modify the route of administration as required to obtain the maximal therapeutic effect.

### EXAMPLES

The following examples are presented for the purpose of illustration only and are not intended to be limiting.

### Example 1: Methodologies to Study CD40-CD40L and Epilepsy

Mice:

Adult male mice (23-33 grams) included CD40 receptor deficient (CD40KO) mice (B6.129P2-Tnfrsf5^{tm1kitk}, from the Jackson Laboratory) and C57BL/6 as wild type control (WT, also from the Jackson Laboratory).

PTZ Model:

Throughout the procedure, mice were placed in individual cages. Pentylenetetrazol (PTZ) (Sigma, St. Louis, Mo.) was administered for induction of seizures. A solution of PTZ, a γ-aminobutyric acid subtype A (GABAA)-receptor antagonist, was prepared in normal saline so that an intraperitoneal injection of 0.25 mL would provide 10 mg/kg PTZ. Mice received injections every 5 minutes until the onset of the first retropulsive myoclonus, defined as a myoclonic jerk resulting in backward movement of the head and shoulders. Afterwards, the animal was euthanized. The latency to the onset of myoclonic jerk was the primary metric recorded. Seizures were classified according to the Racine scale (Musto, 2015) Mice were observed continuously with data recorded regarding time to achievement of respective Racine stage and duration of seizures.

Immunohistology:

Specimens from human TLE, sectioned and mounted in slides, coded, with no identifier and used only for research purpose were obtained from clinical sources. Brains from mice were removed immediately after euthanization, and fixed in formalin 4%, transferred in PBS, followed by 30% sucrose. Coronal 30-μm thick sections were collected for CD40 (CD40, 1/500, sc-20010 Santa Cruz, Inc.) validation of antibody.

Mice brain tissue was collected after using Isoflurane for euthanasia. Each hemisphere was separated and purposed for either IHC or western blotting. Cerebral cortex and the hippocampus of one hemisphere were snap frozen and saved for western blotting. Half of the brain was purposed for IHC and submersed in 4% Paraformaldehyde (FD Neurotech, MD, USA) over a period of 7 days. After 7 days, samples were submersed in a 20% Sucrose solution in Phosphate-buffered saline (FD Neurotech, MD, USA) over 72 hours. Using Mouse/Rabbit Polydetector HRP/DAB kit and following protocol described in (Musto et al., 2006) and provider recommendations, the sections were rinsed in water, dehydrated with ethanol, placed in xylene, mounted, and cover slipped. Cortex, dentate gyrus, CA1 and CA3 regions from the right hippocampus were examined by using standard light microscopy with a Zeiss imaging microscope system. Using a Leica CM1950 Cryostat, 20 micron-thick sections were cut and dried overnight. After slides were dry, they were rehydrated using Xylene and ethanol baths. Slides were treated with serum and other protein complexes to block nonspecific binding and were incubated overnight with multiple primary antibodies (CD40, GFAP, etc.). Slides were washed with PBST and were incubated with a Horseradish Peroxidase conjugated secondary antibody. After at least an hour of the secondary incubation, slides were washed with PBST and were stained with 3,3′-Diaminobenzidine which developed the cell type isolated with the primary antibody. Imaging was performed using an Olympus microscope.

Western Blotting:

Samples that were snap frozen after dissection were placed in a −80° C. refrigerator until Western blotting was performed. To prepare the lysates, about 300 mg of tissue (cortex and hippocampus) was placed in 500 uL of RIPA buffer. A homogenizer was used on the samples multiple times until the tissue disintegrated as much as possible. Samples were then agitated in an orbital shaker for 2 hours at 4° C. Samples were homogenized once more and then placed in a centrifuge at 4° C. and set to 12,000 RPM for 20 minutes. The supernatant was then aliquoted. Bradford Assays were performed to quantify the protein concentration in each lysate. Using the Mini-Protean Tetra Cell assembly for SDS-PAGE (Biorad, California), along with Mini-Protean TGX 4-20% gels, samples were then loaded into their respective lanes, accounting for about 50 ug of protein per lane. While analyzing CD40 and CD40L, the protein concentration was increased to about 100 ug since CD40 and CD40L expression is expected to be low. Gels ran at 100V over approximately an hour. Afterwards, transfer to the nitrocellulose membrane was performed using a cassette set-up. Transfer was performed using 100V over an hour. Nitrocellulose membranes after transfer were then hybridized with the primary antibody in a blocking solution that contains BSA and TBST, set overnight. Membranes were then washed three times prior to secondary antibody hybridization and three more times after. Imaging was performed using the Licor Odyssey system (Li-Cor, State, USA).

Status Epilepticus:

Study mice were pretreated with Scopolamine injections intraperitoneally (IP) (1 mg/kg, IP), 30 minutes prior to status epilepticus induction. Subsequently, Pilocarpine Hydrochloride (280 mg/kg, IP, Sigma Aldrich) was injected and mice were observed over a period of four hours to ensure normal health status. Control mice were injected with equal amounts of sterile saline intraperitoneal. During the first post-pilocarpine observational period (2-4 hours after Pilocarpine), mice were evaluated to assess the development of the status epilepticus using the Racine scale. Any mice that reached Stage 5 were excluded from the study. After the mice recovered at least 2 hours post-pilocarpine, Diazepam (10 mg/kg, IP) was administered as provided by the veterinarian staff from the Comparative Medicine Department (EVMS). Control mice received scopolamine and sterile saline (sham). Mice were left in their appropriate acrylic cages with feed and water over the following 24 hours. Mice were monitored every 8 hours during the 24-hour observational period to ensure healthy levels of hydration and activity.

AntiCD40L Administration:

Using the InVivoMab Anti-Mouse CD40L (CD154) antibody blocks CD40-CD40L interaction in vivo as previously reported (Aarts et al., 2017; Shock et al., 2015). The molecule was validated by Bio X Cell. For intranasal administration, the InVivoMab Anti-Mouse CD40L was diluted to a concentration of 2 mg/mL. The initial concentration was 6.69 mg/mL, and about 300 microliters of antibody solution was diluted into about 700 microliters of sterile saline. The solution was stored in an Eppendorf tube and kept at 4° C. until it was used. At the time of the experiment, 5 microliters of the 2 mg/mL CD40L in sterile saline solution was administered to each naris.

Statistical Analysis:

CD40 positive cells of different morphologies per field (40X) were semi-quantified. Statistical comparisons were conducted for the behavior scale to obtain means and standard errors of the mean (SEM) by using ANOVA and Student's t test for statistical significance (p<0.05). The line through the box represents the median, majority of the data falls between the ends of the whisker, individual horizontal lines: standard deviation, horizontal line mean error bar. These plots were generated with JMP trial software (wwwjmp.com).

### Example 2: CD40 is Expressed in Temporal Lobe Epilepsy

The expression of CD40 is very low in brains of healthy humans, as evidenced by expression data obtained in a public database (The Human Protein Atlas) available under https://www.proteinatlas.org/ENSG00000101017-CD40/tissue. Specifically, FIG. 2A1 shows neurons in a healthy human hippocampus with very weak CD40 immunoreactivity. FIG. 2A3 shows CD40 immunoreactivity in neurons from a patient with glioblastoma multiforme (GBM); the arrow points to the cell body of a neuron. Interestingly, CD40 expression in the hippocampus of the GBM patient, as shown in FIG. 2A3, is stronger than the expression in a healthy human brain, as shown in FIG. 2A1.

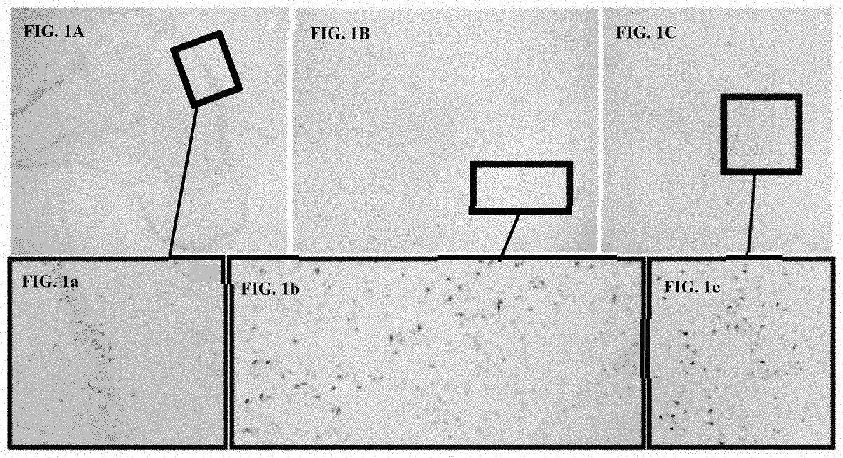

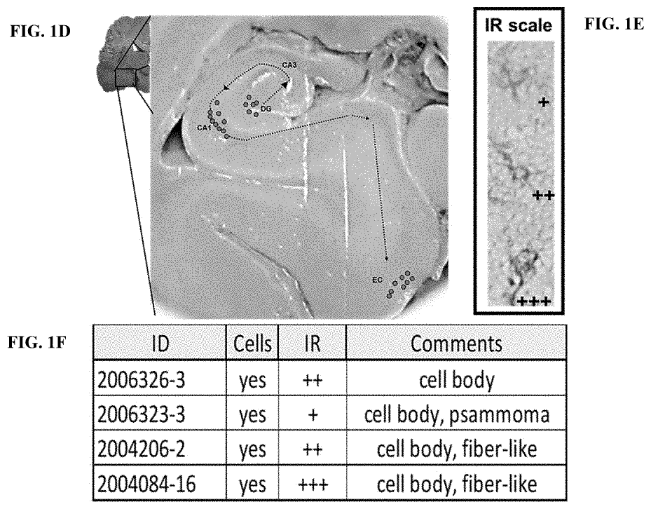

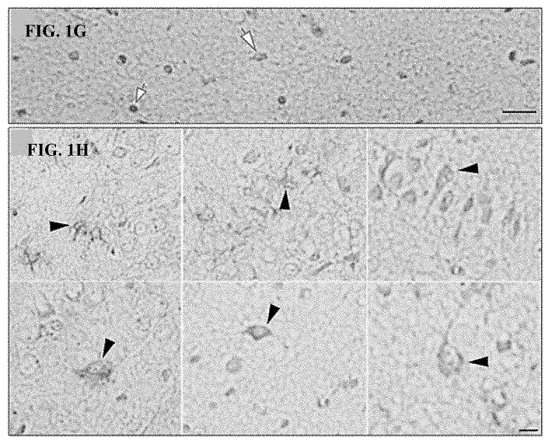

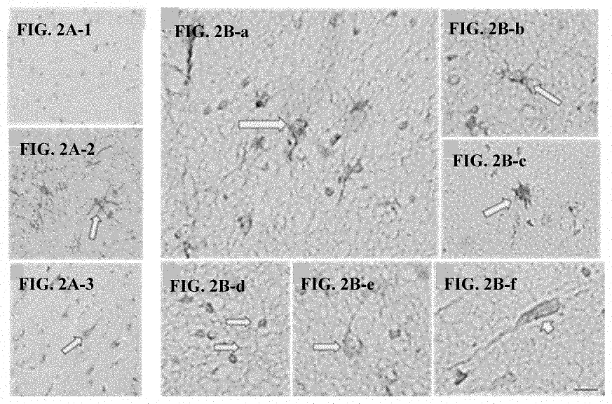

Using immunohistochemical staining and cresyl violet staining of hippocampal cryosections obtained from patients who underwent neurosurgical treatment for Temporal Lobe Epilepsy (TLE), CD40 was found to be highly expressed in the hippocampal region of TLE patients. The increased CD40 expression was associated with neuronal loss in the dentate gyrus and CA1 region of the hippocampus. FIGS. 1A-C are cresyl violet stained histological sections of the hippocampal region from a TLE patient. The upper panel (1A-1C) is displayed at a lower magnification, with FIG. 1A showing dentate gyrus (DG) and FIGS. 1B and 1C showing the CA3 region inside the respective boxes. The areas in the boxes are further magnified in the lower panel in 1a-1c. FIGS. 1A and 1a demonstrate cellular dispersion and loss of neurons; FIGS. 1B-1C and 1b-1c demonstrate disruption of cellular alignment and loss of neurons. These pathological features are representative of TLE type I-II, where neuronal loss and gliosis are predominant in the CA1 subfield including dentate gyrus (Thom, 2014).

A specific antibody against CD40 stained cells that resemble astrocytes, neurons, microglia, and oligodendrites, of patients with TLE (FIGS. 1G, H, FIG. 2). FIG. 1D is a representation of the CD40 immunoreactivity (IR) (dots) distribution in a temporal lobe specimen from a TLE patient, using the same antibody. FIG. 1E depicts a semi-quantification scale to describe CD40 IR. Applying this scale, FIG. 1F summarizes the CD40 IR in the hippocampal tissue of four different TLE patients (referred to with different ID numbers in the left most column). All four patients demonstrated CD40 immunoreactivity localized in cell bodies.

FIGS. 1G (CA1-subicular region) and 1H (other CA1 regions) include sections from TLE patients that demonstrate CD40 IR in human TLE. The white arrowheads in G and black arrowheads in H all point to CD40 IR signals. The white arrowheads in G point to cell bodies of likely neurons; the black arrowheads in H point to cell bodies of astrocytes and neurons.

FIG. 2B provides further sections of hippocampus from a TLE patient with CD40 immunoreactivity (arrows) in astrocytes (a,b), microglia (c), oligodendrites (d) and neurons (e,f). CD40 has an increased expression in the hippocampus of the TLE patient, compared to the hippocampus of a healthy person in FIG. 2A1.

### Example 3: CD40 and CD40L Expression Increased after Seizures

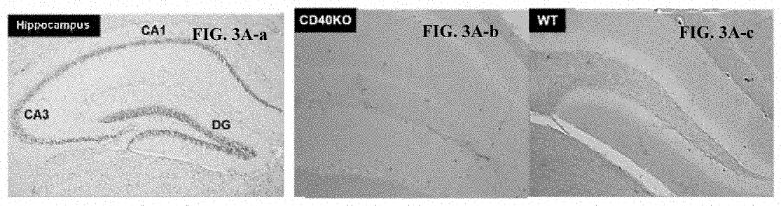

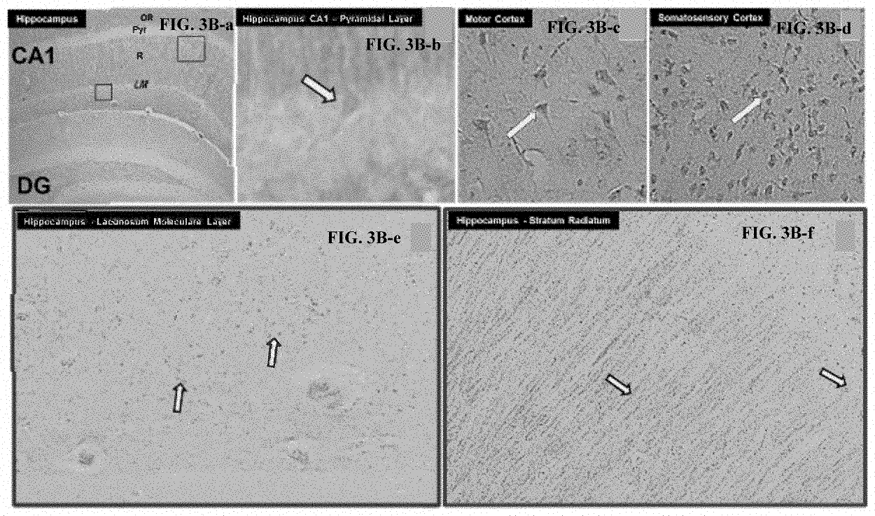

CD40 is expressed in neurons from the neocortex and hippocampus in adult mice (Tan et al., 2002). However, expression of CD40 in the hippocampus is relatively low compared to CD40L (Carriba and Davis, 2017). CD40 IR was shown in the brain of the wild type C57BL/6 mice, especially in hippocampal regions as fiber-like patterns and in cortical neurons (FIG. 3). As a comparison, it is not present in the CD40KO mice (experimental negative control).

FIG. 3 provides representative brain sections from a naïve (no prior seizure) adult mouse. FIG. 3A-a is a coronal section of the dorsal hippocampus of a wild type mouse with no seizure history; it shows dentate gyurs (DG), CA1, and CA3 stained with cresyl violet. FIGS. 3A-b and 3A-c are from similar hippocampal locations and stained with a hamster monoclonal anti-mouse CD40 antibody (sc-20010, Santa Cruz). FIG. 3A-b is from a homozygous CD40 knockout mouse (CD40KO), while FIG. 3A-c is from a wild type mouse (WT). The CD40 expression is higher in the wild type mouse (WT—FIG. 3A-c) than the CD40 deficient mouse (CD40KO—FIG. 3A-b).

FIG. 3B shows details of CD40 expression in the hippocampus of WT mice. FIG. 3B-a provides an overview of different hippocampal CA1 subregions, including OR (Stratum Oriens), Pyr (Pyramidal Layer), R (Stratum Radiatum), and LM (Stratum Lacunosum Moleculare, or Lacunosum Moleculare Layer). The two squares in FIG. 3B-a are further magnified in FIG. 3B-e (the lower left square, Lacunosum Moleculare Layer) and FIG. 3B-f (the upper right square, Stratum Radiatum). FIGS. 3B-b to 3B-d magnify other subregions: FIG. 3B-b depicts Hippocampus CA1—Pyramidal Layer; FIG. 3B-c depicts the Motor Cortex; and FIG. 3B-d depicts the Somatosensory Cortex. In all figures, the sections were stained with a hamster anti-mouse monoclonal CD40 antibody (sc-20010, Santa Cruz). A fiber-like expression pattern can be seen in FIG. 3B-f in Stratum Radiatum; whereas cell bodies constituted most of the staining in motor and somatosensory cortex. Arrows in FIG. 3B point to cells demonstrating CD40 immunoreactivity.

The distribution of CD40 in mouse brain tissue was confirmed by western blot analysis, which indicated a higher level of CD40 expression in the cortex compared to the hippocampus.

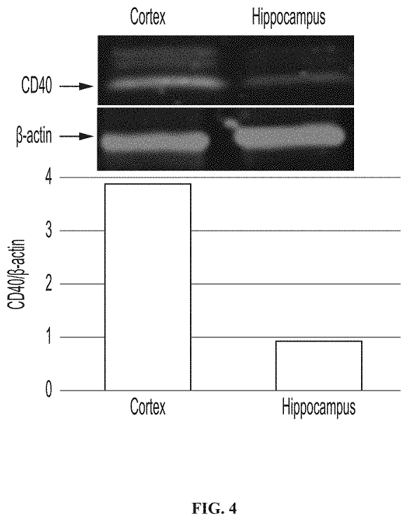

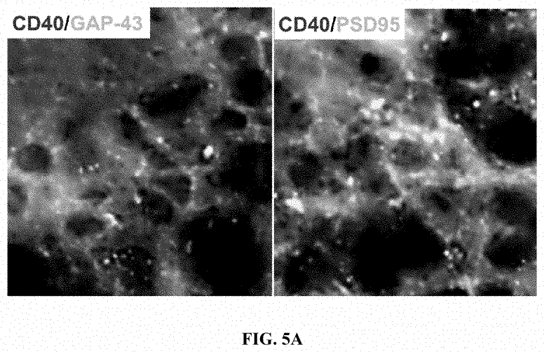

FIG. 4 indicates a higher expression of CD40 in mouse cortex than in mouse hippocampus, based on western blots results. The bar plot shows CD40 immunoreactivity density relative to (3-actin (control) and indicates a significantly higher expression of CD40 in cortex (n=3) compared with hippocampus (n=3).

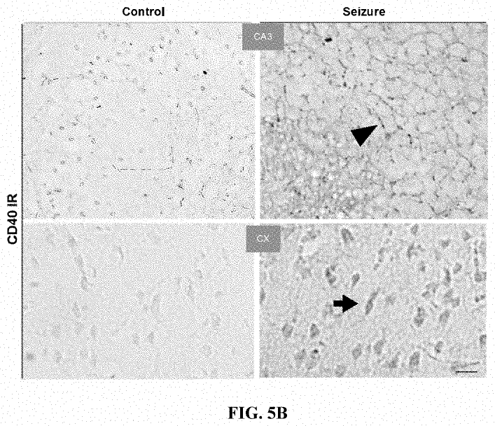

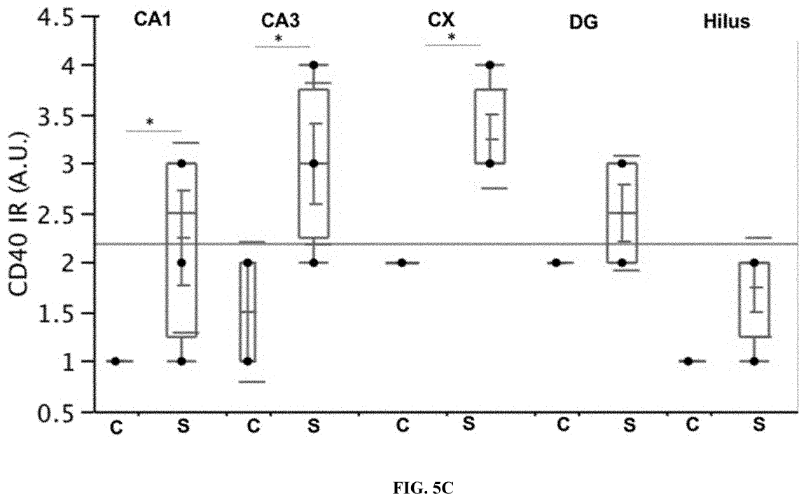

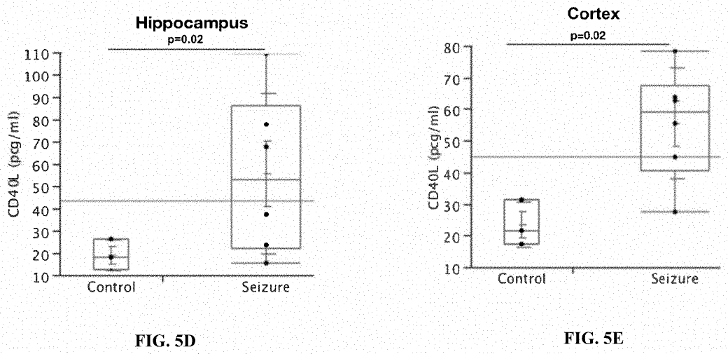

CD40 was found to be expressed in neural terminals. FIG. 5A shows that CD40 is co-expressed with representative presynaptic (GAP-43) and postsynaptic (PSD-95) terminals in the hippocampus, as we observed. The distribution of the CD40 immunoreactivity (IR) was more dense in postsynaptic terminals than presynaptic terminals of naive adult mice.

FIG. 5B depicts CD40 immunoreactivity (IR) in CA3 and somatosensorial cortex (CX), of naïve (without prior seizure) (“control”) and post-seizure (“seizure”) mice, respectively. Tonic seizures were induced through systemic administration of PTZ. The arrows point to exemplary neurons shown to express CD40. Overall, CD40 expression was much higher in the post-seizure brain. CD40 IR was upregulated in neurons of both the somatosensory cortex (Seizure vs. Control p=0.0015) and the hippocampus (Seizure vs. Control p=0.02). After seizure, the CD40 IR increase lead to a fibrillar pattern (probably due to accumulated CD40 in the dendrites) in CA3. In the cortex, however, CD40 IR was increased in the neural cell bodies. The expression in the neurons of the somatosensory cortex appeared to be higher compared to the hippocampus CA3 region.

FIG. 5C is a semiquantitative analysis of CD40 IR in naïve (“c” on the x-axis) and post-seizures (“s” on the x-axis) mouse brains (n=4), based on Image J analyses of histological sections. The hippocampal subregions CA1, CA3, cortex (CX), dentate gyrus (DG), and the hilus are separately examined in this chart. The expression of CD40 IR was significantly upregulated after seizure in CA1, CA3 and in the cortex (CX) (*=p<0.05), but less so in the Hilus and DG (p=0.005).

FIGS. 5D and 5E are semi-quantitative ELISA analysis of CD40 ligand, CD40L in the naïve (control) and post-seizure (seizure) brain. CD40L expression increased in both the hippocampus (FIG. 5D) and the cortex (FIG. 5E) after seizures (n=6) compared to controls (n=3). Therefore, CD40 IR was associated with an increase in the concentration of its ligand, CD40L, in the hippocampus (Control: mean 19.01, ±3.93 S.E.M., n=3 vs., Seizure: mean 55.53; ±14. 67 S.E.M., n: 6; p=0.02) and cortex (control: mean=23.79; ±4.14 S.E.M.; n:3 vs., Seizure: mean:55.40±7.16 S.E.M., n=6; p=0.02) respectively (FIGS. 5D and E).

### Example 4: CD40L-CD40 Concentration was Higher 24 Hours after Status Epilepticus

Neuroinflammation plays a critical role in the development of epilepsy during the acute phase of epileptogenesis, approximately 24 hours after SE in the pilocarpine model of TLE. Using an enzyme linked immunosorbent assay (ELISA), CD40L and CD40 were assessed after SE. Considering that CD40L-CD40 interaction is key in activating an inflammatory process, the relationship of the concentration of CD40L and CD40 in brain was evaluated by analyzing an index of CD40L over CD40 concentrations.

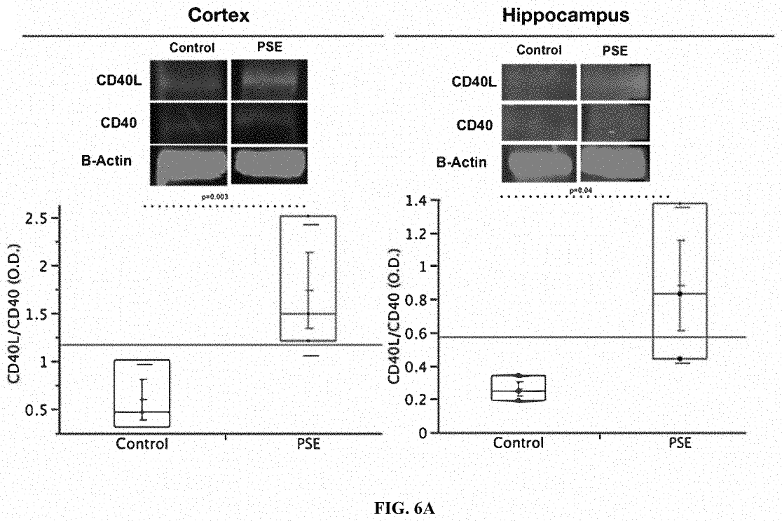

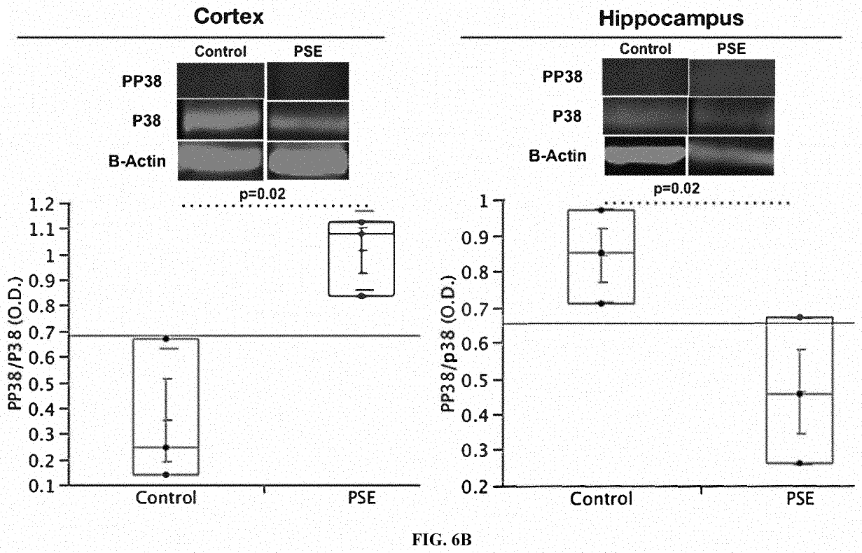

FIG. 6 demonstrates that CD40 and CD40L both increase after status epilepticus. The upper panel of FIG. 6A shows that the expression of CD40L and CD40 in the cortex and the hippocampus was higher in mice post status epilepticus (PSE) than in naïve mice (control). It was observed that the ratio of CD40L/CD40 increased in both cortex (Control: mean: 0.5, ±0.2, S.E.M., n=3 vs. PSE: mean: 1.7, ±0.3 S.E.M.; n=3; p=0.003) and hippocampus (Control: mean: 0.25, ±0.043 S.E.M.: n=3 vs. PSE: mean:0.88, ±0.27 S.E.M.: n=3; p:0.042) after SE (FIG. 6A). Therefore, although the expression of CD40 and CD40L both increased, CD40L increased even more.

Additionally, the p38 MAP kinase participates in CD40 signaling pathway and has been implicated in epilepsy via a c-Jun N-terminal kinase. During the acute phase of epileptogenesis, the relationship between pp38 (phosphorylated p38) and p38 increased in cortex (Control: mean: 0.35, ±0.16 S.E.M., vs. PSE: mean:1.01, ±0.15 S.E.M., p=0.01) but decreased hippocampus (Control: mean: 0.8, ±0.07 S.E.M., n=3, vs. PSE: mean: 0.46, ±0.11 S.E.M.; n=3, p=0.03) (FIG. 6B).

### Example 5: Monitoring Clinical and Electrical Seizures in Freely Moving Mice During Epileptogenesis

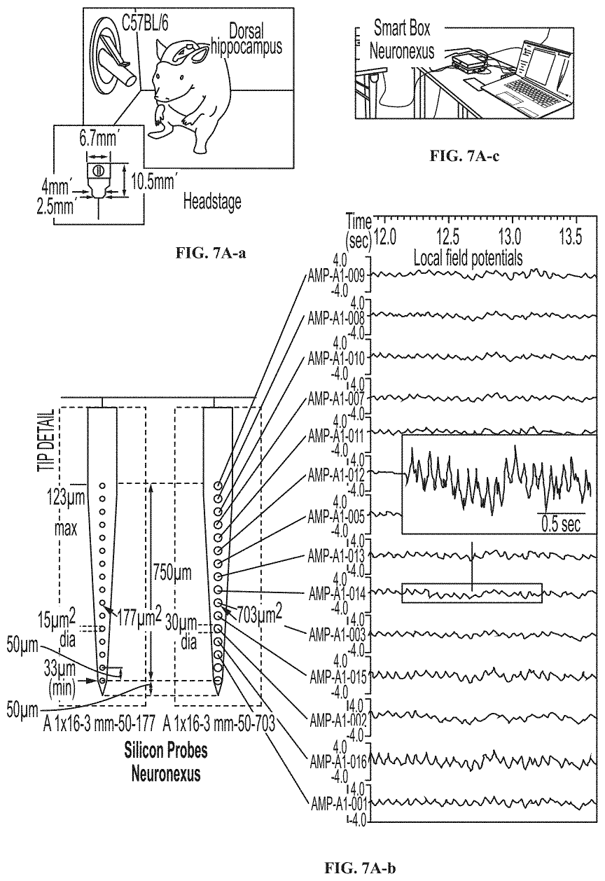

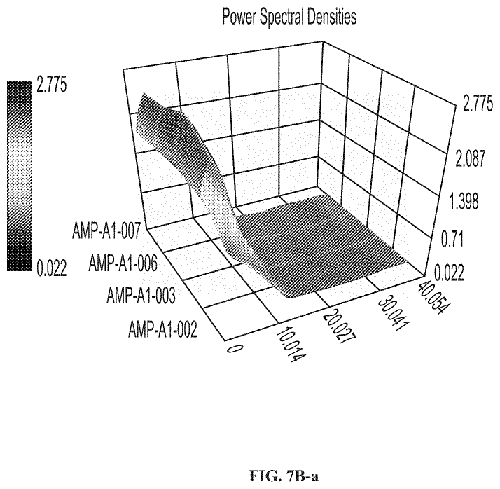

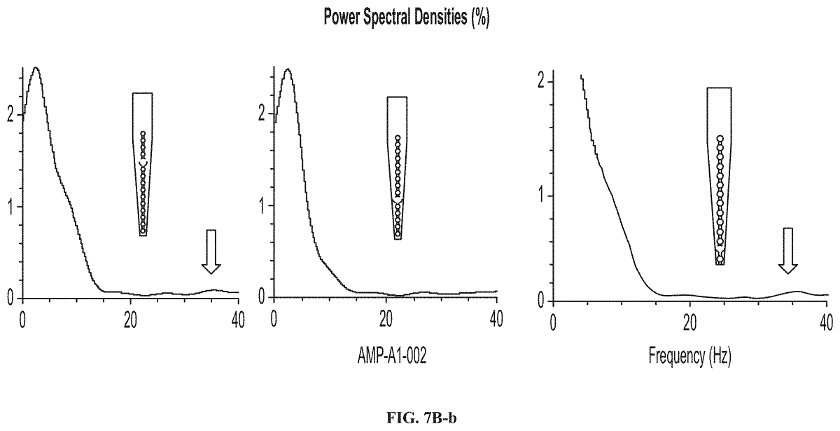

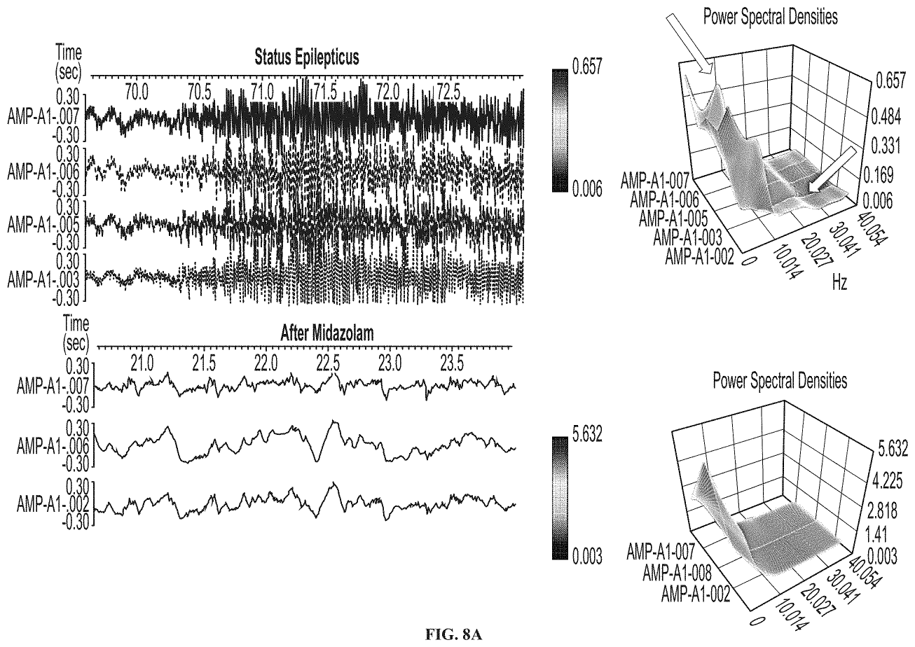

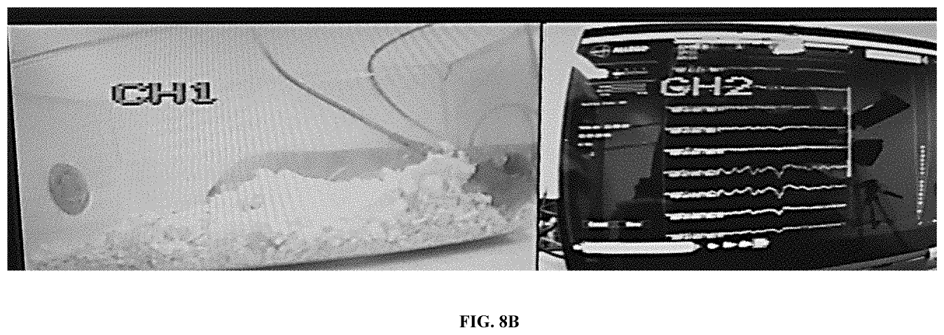

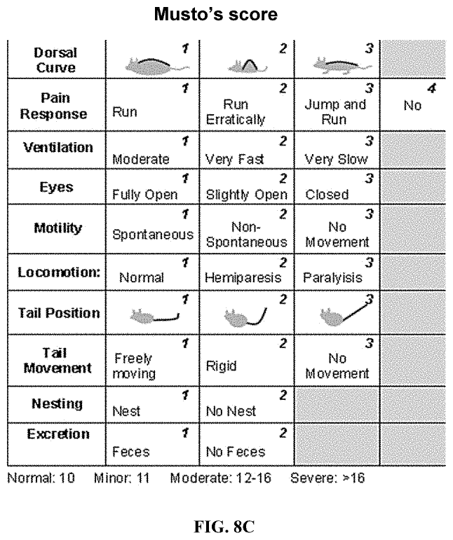

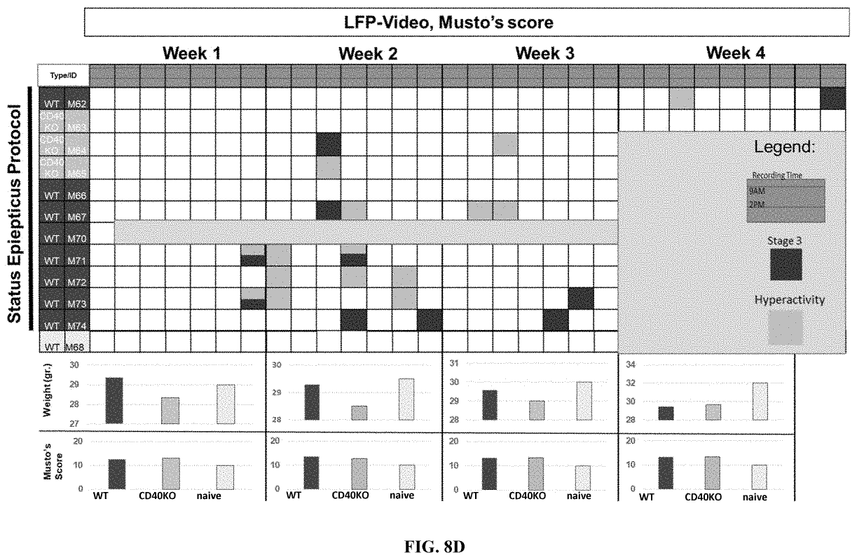

It has been found that in vivo hippocampal local field potentials (LFPs) from freely moving mice during epileptogenesis present spontaneous micro epileptiform activities in CA1 and DG (Musto et al., 2015, Musto et al., 2016) that can predict the onset of epilepsy. Freely moving animals were monitored to study physiological changes, seizures, and aberrant activities in epileptogenesis (FIGS. 7 and 8). A silicon probe was implanted in the dorsal hippocampus of mice under anesthesia. The surgery was successfully accomplished with 100% survival of the mice selected. Then animals were placed in a behavior room to record local field potentials at different time points after surgery. One week after the initial induction of seizures through chemical means, dorsal hippocampal activity showed aberrant physiological oscillations at rest, sleeping, and walking. FIG. 7A-a is a photograph of a C57BL/6 mouse showing a silicon probe attached with acrylic cement on its head 24 hours after surgery. FIG. 7A-b is an example of a silicon probe design showing the assembled 16 microelectrodes and their local field potentials. FIG. 7A-c is an example of a Neuronexus smart box for digital recording of local field potentials (LFPs). Other methods of recording local filed potential may also be used.

FIG. 7B-a represents overall oscillatory activity using power spectral analysis showing physiological profile at different region of hippocampus. There was an increase of theta activity in all regions, and an increase of gamma activity in the most dorsal and ventral channels. The graphic depicts power spectral density (PSD) from 0.022 to 2.77 (see left scale) between different channels AMP-A1.002, 0.003, 0.006 and 0.007 from silicon probe and frequencies from 0, 10.014, 20.027, 30.041 and 40.054. FIG. 7B-b depicts PSD (Y axis) vs frequencies (X axis) Hz from 3 different electrodes (filled dot) in the silicon probe. Theta waves were observed between 4-12 hertz, while gamma waves are seen at around 30 hertz.

The pilocarpine model of epilepsy (Musto et al., 2015) was used to induce epileptogenesis. Briefly, this model was used to induce status epilepticus (SE) by intraperitoneal administration of pilocarpine, followed by intraperitoneal administration of midazolam after 90 minutes of SE, then animals that survive are expected to develop spontaneous recurrent seizures 14-20 days later. This model allowed investigations into the expression of CD40, CD40L, and the inflammation in hippocampus before onset of epilepsy, in addition to studying interventions that block the CD40-CD40L interaction during the first week after SE.

To determine the concentration of CD40 and CD40L, the cellular damage and inflammation during epileptogenesis and epileptic state, 12 mice were studied under the SE protocol (FIG. 8A) with control (no pilocarpine admiration) animals included in the experiment for comparison. FIG. 8A shows a representative LFPs recording during Status Epilepticus (SE) and after midazolam, demonstrating the respective overall oscillatory activity, the increased frequencies in SE and the attenuation after midazolam administration. The corresponding Power Spectral Densities plots are shown next to the LFPs.

All animals survived; their weight and physiological conditions (using the Musto scoring scale) were recorded twice per week over 21 days (FIGS. 8C and D). Video recording of the spontaneous behavior was conducted at least 6-8 hours/day consecutively for 21 days after SE. In mice with implanted silicon probes, local field potentials and behaviors were recorded simultaneously (FIG. 8B) up to 21 days after SE. Most of the mice showed hyperactive locomotor behavior during the second week after SE.

FIG. 8C is a description of Musto's score. This score was based on an assessment of various characteristics in a rodent, such as posture, locomotor activity, ventilation, pain responses, aperture of eyes, spontaneous tail movements, nesting and number of feces in the cage. Musto's score was used to evaluate the mice being recorded and observed for LFP and behavior. FIG. 8D is a table showing spontaneous seizures and hyper-activity after status epileptics (SE). Mice with deficiency of CD40 (CD40 KO mice, M63, M64, and M65) showed a lower frequency of seizures compared with wild type mice post SE (WT, M62, M66, M67, M70, M71, M72, M83, M74) and WT mice that had not experienced SE (M68). Bars below table depict animals showing a minor to moderate Musto's score (Normal: 10; Minor: 11, Moderate: 12-18; Severe: >16) after SE with no significant weight loss over 4 weeks. For each 3-bar group, the left most bar represents WT mice post SE, the middle bar represents CD40 KO mice post SE, and the right most bar represents WT naïve mice.

### Example 6: CD40 Deficiency Attenuates Seizure Susceptibility

A pentylenetetrazol (PTZ)-induced seizure model was used to determine if the presence of CD40 promotes seizure susceptibility. Successive sub-convulsive doses of PTZ were administered to determine the threshold for different types of seizures using Racine's score. Adult male CD40KO mice (25-30 g) (n=7) and respective age-gender controls (WT, n=7) received intra-peritoneal PTZ administration (10 mg/Kg) every five minutes until either the animals elicited tonic-clonic seizures or received a total of 60 mg/kg of PTZ. Then, animals were euthanized, and the brain samples were processed for histological analysis. Seizure severity (Racine's score), latency, and seizure frequency were analyzed using student's T-test.

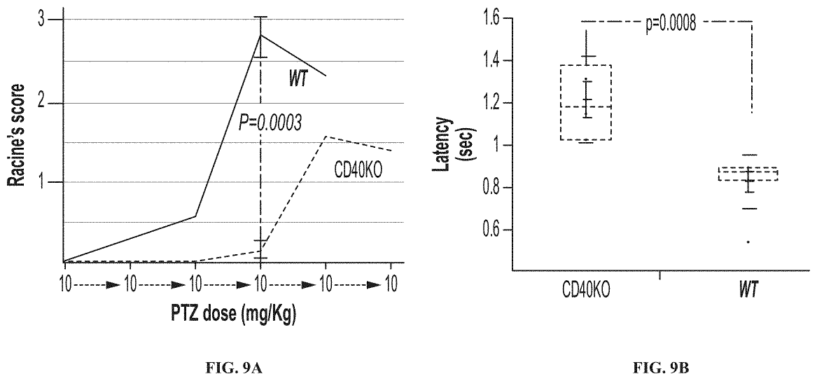

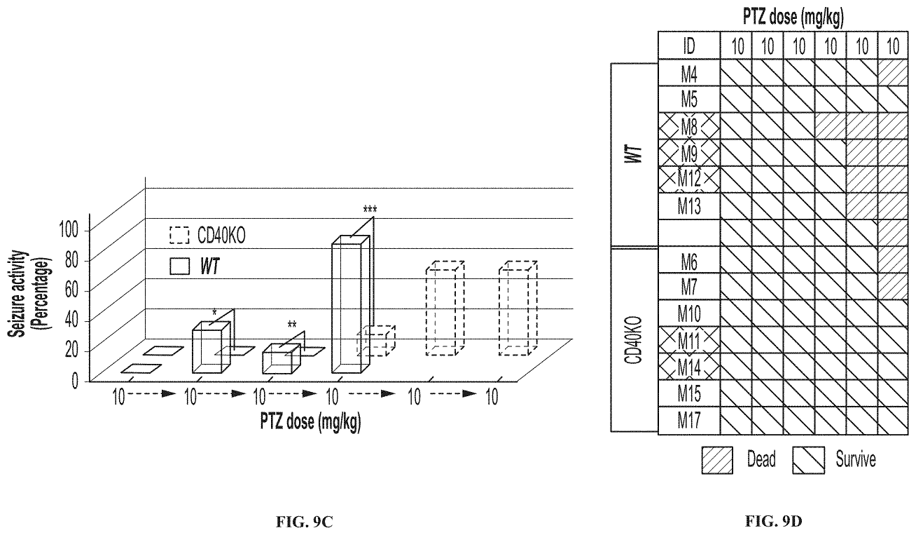

FIG. 9A is a graph illustrating that CD40KO mice present a remarkable reduction of seizure severity at 40 mg/kg intraperitoneal doses of pentylenetetrazol (PTZ) compared to WT mice. CD40KO mice exhibited reduced seizure occurrence compared to WT, with statistically significant differences at 40 mg/kg (Racine's score of the CD40KO mice: 0.1+−0.14 SEM; Racine's score of the WT mice: 2.8+−0.54 SEM; p=0.0003). In addition, FIG. 9B illustrates that CD40KO demonstrated increased latency in seizure onset at 50 mg/kg compared to WT (CD40KO: 4.45 minutes±0.51 S.E.M vs. WT: 1.09 minutes±0.005 S.E.M. p=0.0008), whereby reaching peak Racine's score of 2.71 after 50 mg/kg compared to WT with 3.42 at 40 mg/kg. Overall, CD40KO mice showed a reduction in seizure frequency compared to WT mice at various doses of PTZ, especially at 20, 30, and 40 mg/kg (CD40KO: 0, 0, 14.28; WT: 28.85, 14.28, 85.7; p=0.0075; p<0.0001; p=0.05, respectively) (FIG. 9C). Finally, CD40KO mice exhibited lower mortality compared to WT mice (FIG. 9D). These results indicate that CD40 may play a role in propagating seizure development, which was unexpected and surprising. CD40L-CD40 has not previously been reported in human epilepsy.

### Example 7: CD40 Deficiency Limited Severity of SE

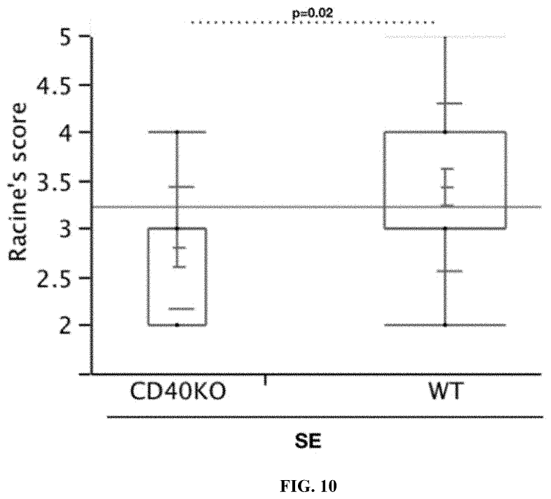

FIG. 10 demonstrates that CD40 deficiency limits the severity of Status Epilepticus. CD40KO mice present lower Racine's score compared to WT mice during SE induced by pilocarpine. CD40KO mice exhibited seizures with reduced severity than WT (Racine's score in CD40KO: 2.8±0.2 S.E.M. n=10 vs. WT: 3.42±0.18 S.E.M. n=21; p=0.02) in the induced SE after pilocarpine administration. CD40 deficiency also prevented mortality following SE. (CD40KO: 0 dead vs. WT: 4 dead). Therefore, CD40 deficiency mitigated the severity of status epilepticus, which was unexpected and surprising. CD40L-CD40 has not previously been reported in human epilepsy.

### Example 8: Characterization of CD40-CD40L Expression in Epilepsy

To further assess the expression of CD40-CD40L in epilepsy and evaluate potential neuroprotective and/or anti-inflammatory function of an antibody against CD40L, cellular modifications after different types of epileptic conditions were determined. FIG. 11 provides a cresyl violet stained coronal section of dorsal hippocampus showing hippocampal subregions CA1, CA3 from naïve WT mice (control), CD40KO mice and WT mice at three weeks after Status Epilepticus (Post SE-3 weeks), respectively.

Cell loss was immediately observed after tonic-clonic seizures (Racine's score 4) induced by PTZ or 24 hours after SE induced by pilocarpine (FIG. 11). Those changes were mainly located in hilus, CA1, and CA3. The CD40KO mice demonstrated better preservation of neuronal cells in these hippocampal subregions compared with WT mice, where the oval circles highlight areas with loss of neurons.

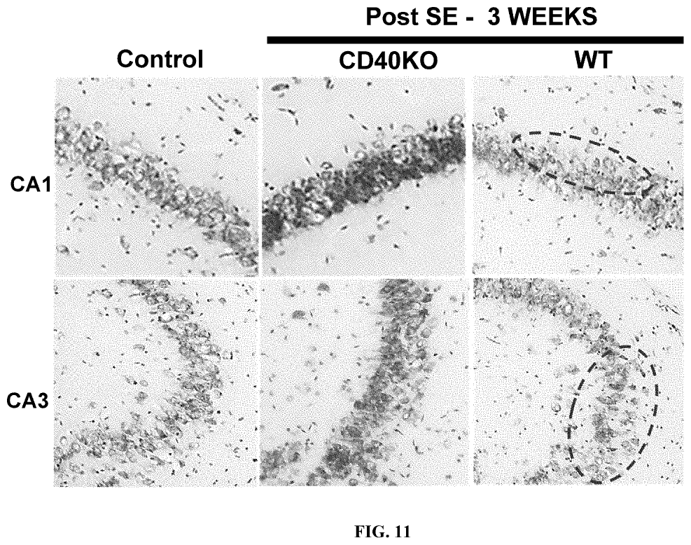

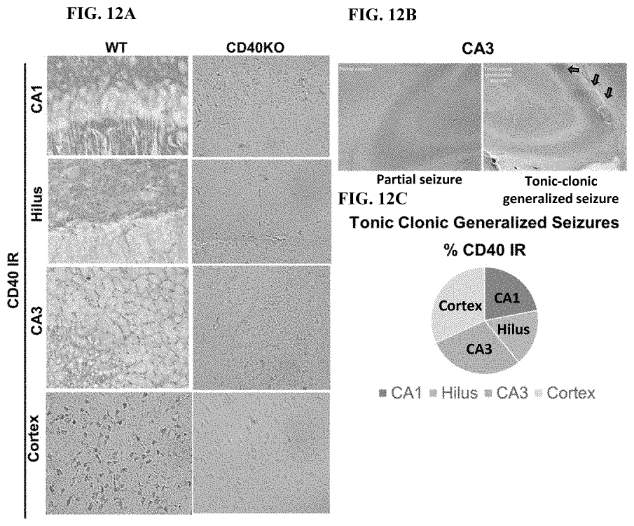

FIG. 12 shows coronal sections of dorsal hippocampus and somatosensory cortex from WT (C57BL/6) and CD40KO mice (negative control for WT mice) after seizures induced by PTZ. FIG. 12A shows that, in cortex, CD40 was expressed in the cell bodies. In the hippocampus, however, the CD40 IR showed a fiber-like profile. FIG. 12B shows that CD40 was more prominently expressed in the CA3 region of wild type mice after tonic-clonic generalized seizures than after partial seizures (Racine's score 1-3). The arrows FIG. 12B for generalized seizure point to areas of especially high IR. No such areas existed after partial seizures. FIG. 12C is a chart showing the distribution of CD40 IR in the hippocampus.

### Example 9: Administration of Anti-CD40 Limits Seizure Susceptibility

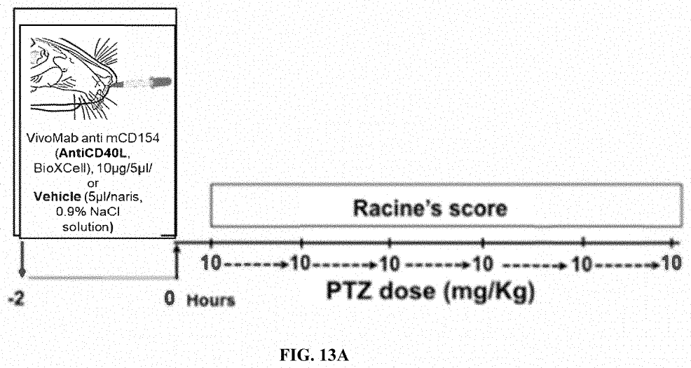

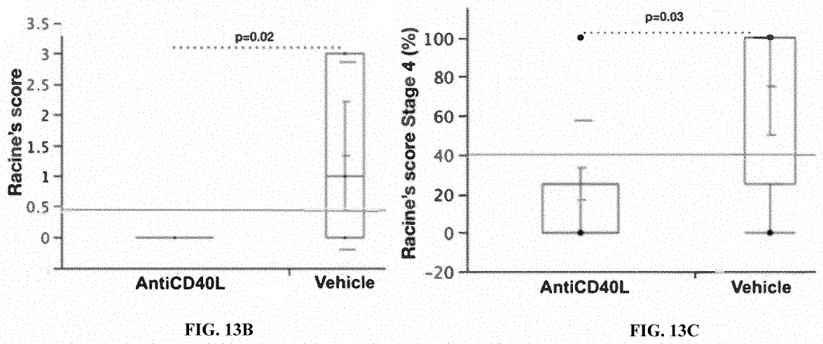

BioXCell InVivoMAb anti-mouse CD40L (CD154), which works as an antagonist of CD40L (Bxcell.com/production/m-cd154-_cd40l_/), was used to block the CD40-CD40L interaction. Since intranasal delivery of anticonvulsive drugs has been postulated in epilepsy, the anti-CD40L antibody was administered intranasally before seizure induction using PTZ. FIG. 13A is the experimental design that was used to test this effect. Anti-CD40L (BioXCell InVivoMAb antimouse CD40L (CD154)) or vehicle (sterile saline) was administered intranasally two hours before seizure induction with PTZ. PTZ was administered every 5 minutes, each time at 10 mg/Kg body weight.

Mice treated with intranasal dosing of anti-CD40L antibody exhibited a reduction in seizures after an accumulative dose of 30 mg/kg of PTZ (Racine's score in Anti-CD40L: 0±0 S.E.M. n=6 vs. Vehicle: 1.8±0.88 S.E.M n=4; p=0.02) (FIG. 13B). In addition, anti-CD40L limited seizure severity after respective doses of PTZ (Percentage reaching Racine's score 4 in anti CD40L: 16%±16. 6 S.E.M. n=6 vs. Vehicle: 75%±25 S.E.M. n=4; p=0.03) (FIG. 13C).

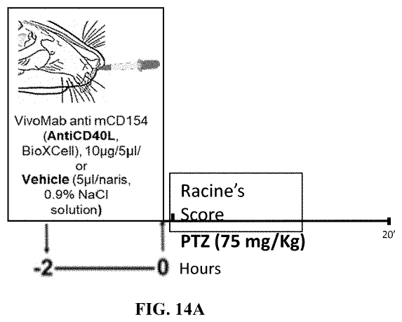

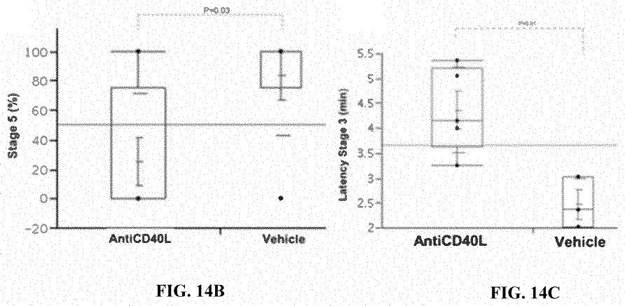

FIG. 14 demonstrates that intranasal administration of anti-mouse CD40L limits severe seizures. FIG. 14A shows the experimental design that involves a one-time administration of 75 mg/Kg PTZ. Anti-CD40 ((BioXCell InVivoMAb antimouse CD40L (CD154)) or vehicle (sterile saline) were administered intranasally two hours before seizure induction with PTZ. Using a single injection of PTZ at convulsive doses, anti-CD40L antibody treated animals showed reduction in percentage reaching tonic clonic seizures (i.e. Racine's score 5) compared to vehicle (Anti-CD40L: 25%±16.36 S.E.M. n=8; vs. Vehicle: 83.33±16.66 S.E.M n=6; p=0.03) (FIG. 14B) and significant increase in latency to reach Racine's score 3 (Minutes in anti CD40L 4.36±0.37 S.E.M. n=5 vs. Vehicle: 2.4±0.2 S.E.M. n=3; p=0.01) (FIG. 14C).

CD40 is expressed at a very low level in normal human hippocampus. It is therefore unexpected and surprising that genetic deficiency in CD40 or intranasal administration of anti-CD40L antibody limited seizure susceptibility, and reduced the frequency and severity of acute seizures induced by PTZ.

As will be apparent to one of ordinary skill in the art from a reading of this disclosure, the present disclosure can be embodied in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein. The scope of the invention is as set forth in the appended claims and equivalents thereof, rather than being limited to the examples contained in the foregoing description. The contents of all of the references disclosed herein are incorporated by reference in their entirety.

### REFERENCES

Engel J Jr, McDermott M P, Wiebe S, et al. Early surgical therapy for drug-resistant temporal lobe epilepsy: a randomized trial. JAMA. 2012; 307(9):922-930.
Sloviter 2008 Sloviter R S. Hippocampal epileptogenesis in animal models of mesial temporal lobe epilepsy with hippocampal sclerosis: the importance of the “latent period” and other concepts. Epilepsia. 2008; 49 Suppl 9:85-92.
Sutula 1989 Sutula, T., Caseino, G., Cavazos, J., Parada, I. and Ramirez, L. (1989), Mossy fiber synaptic reorganization in the epileptic human temporal lobe. Ann Neurol., 26: 321-330.
Musto 2011 Musto A E, Samii M. Platelet-activating factor receptor antagonism targets neuroinflammation in experimental epilepsy. Epilepsia. 2011; 52(3):551-561.
Musto A E, Walker C P, Petasis N A, et al. Hippocampal neuro-networks and dendritic spine perturbations in epileptogenesis are attenuated by neuroprotectin d1. PLoS One 2015; 10: e0116543. Aarts S, Seijkens T T P, van Dorst K J F, et al. The CD40-CD40L Dyad in Experimental Autoimmune Encephalomyelitis and Multiple Sclerosis. Front Immunol 2017; 8: 1791.
Shock A, Burkly L, Wakefield I, et al. CDP7657, an anti-CD40L antibody lacking an Fc domain, inhibits CD40L-dependent immune responses without thrombotic complications: an in vivo study. Arthritis Res Ther 2015; 17: 234.
Thom, Hippocampal sclerosis in epilepsy: a neuropathology review. Neuropathol and Appl Neurobiol. (2014), 40, 520-543.
Tan J, Town T, Mori T, et al. CD40 is expressed and functional on neuronal cells. EMBO J. 2002; 21(4)1643-652.
Carriba P, Davies A M. CD40 is a major regulator of dendrite grog h from developing excitatory and inhibitory neurons. Elife. 2017; 6:e30442.

What is claimed is:

1. A method for treating seizures in a subject in need thereof, comprising administering to the subject a therapeutically effective amount of an agent that inhibits the CD40-CD40L interaction and pathway in the brain.

2. The method of claim 1, wherein the subject has increased susceptibility for epilepsy.

3. The method of claim 2, wherein the increased susceptibility for epilepsy is due to a condition selected from the group consisting of traumatic brain injury, brain tumors, stroke, neuroinflammation, dementia, drug intoxication, chemical intoxications, neurodevelopmental disorders, and metabolic disorders of the nervous system.

4. The method of claim 2, wherein the increased susceptibility for epilepsy is due to a systemic disease selected from the group consisting of diabetes, hypertension, chronic inflammatory disorders, and immunological disorders.

5. The method of claim 1, wherein the subject manifests epilepsy.

6. The method of claim 5, wherein the epilepsy is Temporal Lobe Epilepsy (TLE).

7. The method of claim 5, wherein the epilepsy is status epilepticus.

8. The method of claim 1, wherein the subject does not manifest epilepsy.

9. The method of claim 1, wherein the agent is an antagonist of CD40 or CD40L.

10. The method of claim 9, wherein the antagonist is a CD40L antagonist.

11. The method of claim 10, wherein the CD40L antagonist is an anti-CD40L antibody.

12. The method of claim 11, wherein the anti-CD40L antibody comprises a fully humanized anti-CD40L antibody.

13. The method of claim 11, wherein the anti-CD40L antibody comprises a fragment of a fully functional anti-CD40L antibody.

14. The method of claim 11, wherein the anti-CD40L antibody is PEGylated.

15. The method of claim 11, wherein the anti-CD40L antibody is administered in a pharmaceutically acceptable composition.

16. The method of claim 11, wherein the anti-CD40L antibody is administered by transdermal, transmucosal, intravenous, intramuscular, subcutaneous, intrathecal, intracerebral, intraarterial, intracisternal, endovenous, intraocular, oral or intradermal routes.

17. The method of claim 11, wherein the anti-CD40L antibody is administered intranasally.

18. The method of claim 17, wherein the anti-CD40L antibody is administered as nasal drops or a nasal spray.

19. The method of claim 1, wherein the severity of seizures is decreased after the subject is treated.

20. The method of claim 1, wherein the frequency of seizures is decreased after the subject is treated.
